(19) United States
(12) Patent Application Publication
(10) Pub. No.: US 2025/0180528 A1
(43) Pub. Date: Jun. 5, 2025

(54) SYSTEM FOR COMPREHENSIVE, APPLICATION-GUIDED, HUMAN SENSORY PROFILING

(71) Applicant: Datar Sahi, Marina del Rey, CA (US)

(72) Inventor: Datar Sahi, Marina del Rey, CA (US)

(21) Appl. No.: 18/957,791

(22) Filed: Nov. 24, 2024

Related U.S. Application Data
(60) Provisional application No. 63/604,518, filed on Nov. 30, 2023.

(51) Int. Cl. G01N 33/00; G16H 50/70

(52) U.S. Cl. CPC G01N 33/0001; G16H 50/70

(57) ABSTRACT

The invention is an application-guided, human-senses testing system having tests for all five human senses. A user uses a mobile device to test one's senses and comparative results are provided by a network-lined remote server.

This application seeks priority with its provisional application, application No. 63/604,518 filed on Nov. 30, 2024.

### TECHNICAL FIELD

The invention is a system for measurement of human-sensory performance.

### BACKGROUND OF THE INVENTION

Clinicians have a variety of sensory testing means at hand for individual testing of human senses. These senses are: vision (photoreception), hearing (audition), touch (tactile perception), smell (olfaction) and taste (gustation) senses.

Currently, there are no systems-clinical or non-clinical—that test all five senses, individually, and combine the results into a performance profile score relative to a large aggregation of those tested and their test results.

As a result, there are no systems operative to test all five senses, input the results, combine them, compare them, and produce a relative sensory profile score.

### BRIEF SUMMARY OF THE INVENTION

This invention is a sensory testing system that comprises tests for all the five senses. The tests, guided by a mobile-device-centered application and remote server, are done individually. The individual test results are conveyed to the remote server, and the mobile device receives relative individual sensory scores from the remote server.

After all five tests are completed, and data for each is conveyed to the remote server, the results are processed to produce a comprehensive relative-score sensory profile. The profile can be based on the scores of all who have been tested, and can be further evaluated in terms of filtering results by gender and age, or other variables. As more tests are done, the accuracy of the profiles will increase.

A mobile device, making use its touch screen for output display and input icon selection, is used to provide the full photo reception and audition tests. Additionally, making use of separate olfaction, gustation and tactile perception test subsystems, in coordination with the mobile-device's application, all five senses are measured and scored.

The five tests are based on well-known testing methods. Differences in mobile-device display and sound outputs are compensated through bench-marking normalizations. This reduces differences due to such variables.

Test results are conveyed from mobile device to remote server using mainstream wireless data network conveyance means. Similarly, processed results conveyed by the server to the mobile device also use wireless data network conveyance means.

As results are conveyed to the server, its programmed algorithms compute individual test scores. When all of an individual's test data has been conveyed, the server, using a machine-learning process, continues to refine its weighting so as to improve the model and its accuracy.

### BRIEF DESCRIPTIONS OF THE DRAWINGS

FIG. 1 depicts an embodiment of the invention's mobile-device-centered subsystem's testing structures and functions.

FIG. 2 depicts an embodiment of the inventions remote server engaging with the mobile device.

FIG. 3 depicts an embodiment flow diagram showing process steps.

### DETAILED DESCRIPTION OF THE INVENTION

The invention is operative to provide testing of all five human senses thereby providing a relative score of individual tests and a comprehensive sensory profile based on all five sensory test results.

The invention comprises five subsystems, one that is mobile-device-centered, one that comprises a remote server operative to process incoming test data, calculate scores, and return calculated scores to the mobile-device-centered subsystem, and three test-kit subsystems for smell, taste and touch sense testing in conjunction with mobile device application guidance.

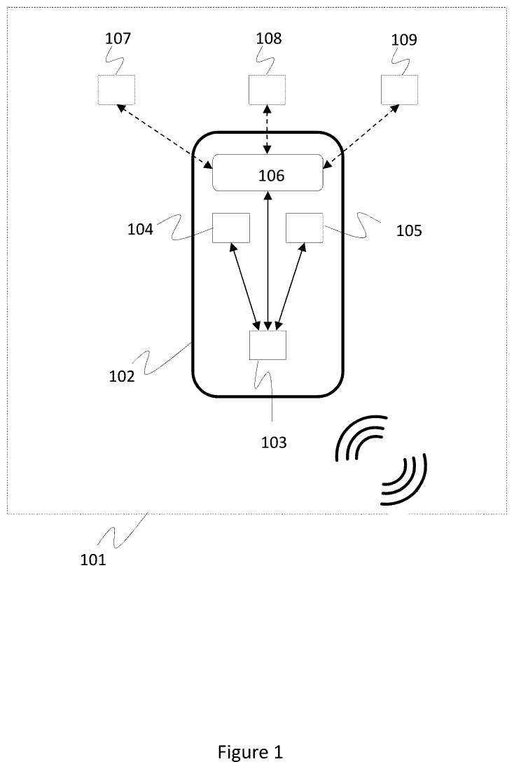

FIG. 1 shows the mobile-device-centered subsystem (101). A mobile device (102) hosts a plurality of program modules and their process algorithms. One module, 103, processes all the test data conveyed to it by the individual testing software modules. For example, the photoreception testing module (104) is operative to send display commands to 103 which module, in turn, controls the touch-screen display and touch selection.

In this exemplary embodiment, module 104 may display side-by-side rectangular bars of essentially the same color but one is slightly different in hue than the others. When a user touches a bar, touch-screen 106 sends the touch data to 103 which then determines if it is correct or incorrect. Process 103 may feedback that interim result to the user.

In a similar fashion audition testing module 105 sends commands to 103 affecting display directives and sounds reproduced. Here, again, as a user touches an icon, the selection choice is conveyed by touch-screen 106 to process module 103 which then determines if the selection and sound are concurrent (e.g. correct or incorrect). Process 103 may again feedback that interim result to the user.

Olfaction, gustation and tactile perception, unlike photo reception and audition, are not dependent on the touch-screen display. External, physical, testing subsystems are used in conjunction with the application's guidance. Directives are displayed, icons are touched, and data is conveyed by touch-screen 106 to the process module 103. Interim results may be shared with the user, or the results can be shared once all test steps have been completed.

As shown in FIG. 1, the interface between olfaction test kit subsystem (107), gustation test kit subsystem (108) and tactile perception test kit (109) are not direct (shown as dotted lines). Instead, for example, with the olfaction test kit subsystem (107), a card containing a plurality of smell test samples, under direction from 103 via 106 instructs the user to scratch and sniff, then choose a smell choice (e.g. leather, soap, rose, banana). The interface is shown as bidirectional because directives conveyed by process module 103 affect the user's actions, and a user's icon selection affects the process module's determination of correct or incorrect.

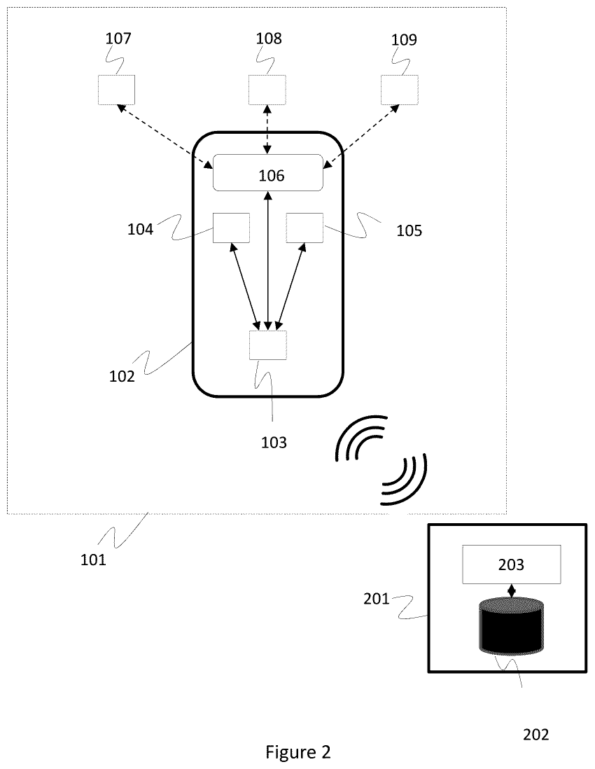

FIG. 2 shows the mobile-device-centered subsystem wirelessly engaged with the remote server subsystem 201. The remote server subsystem comprises a database (202) and a control processor/input-output/wireless interface module (203).

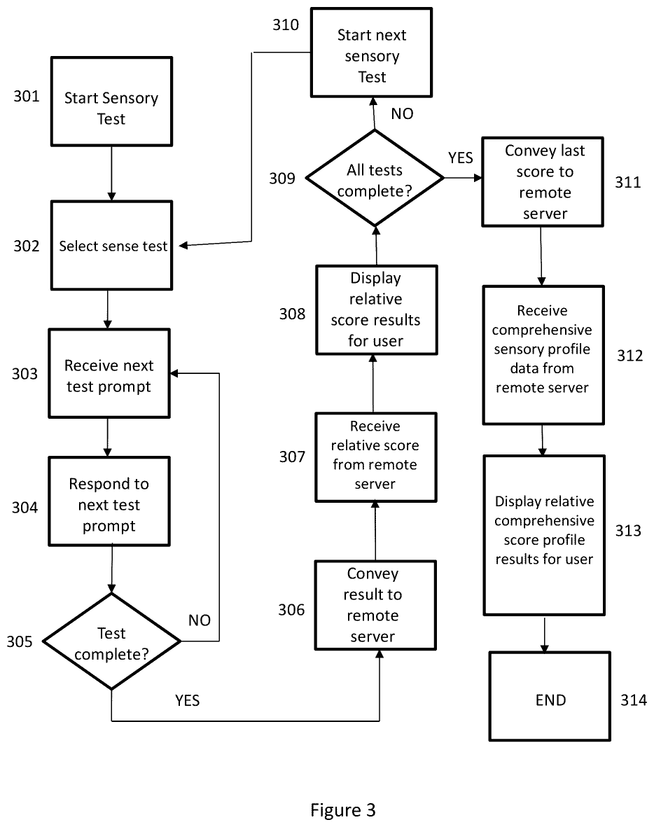

FIG. 3 is an exemplary flow diagram showing a sequence of steps that embody the invention and its use. The flow diagram is not specific to any particular sensory step but covers the eventual sequence of tests and the overall sensory profile score that is produced. It begins (301) with selecting, on the mobile-device-centered subsystem, a sensory test (302) followed by receiving of a prompt (303), responding to that prompt (304) and continuing to repeat that process until a test is completed (305). When completed, the result is conveyed to the remote server (306). The remote server processes the result and produces a relative score which is then conveyed to the mobile-device-centered subsystem (307). The mobile-device-centered subsystem displays the result for the user (308) and determines if all five tests are complete (309). If not, it repeats (310) the preceding steps for successive tests. When all tests are completed, the last score is conveyed to the remote server (311), which then processes the results of all the tests and produces a comprehensive score sensory profile. That profile data is conveyed back to the mobile-device-centered subsystem (312), which in turn displays the comprehensive score profile for the user, and the testing concludes (314).

This embodiment makes use of a mobile-device-centered subsystem as the direct interface to the user in conjunction with a remote server and its hosted processes. The individual tests hosted by the mobile-device-centered subsystem (photoreception and audition) can be updated with modified or new methods and processes. The external tests (olfaction, gustation and tactile perception) may be updated with modified or new methods and processes.

In addition to providing a user with a relative indication of a user's five senses scores and a combinational score, these test results may be associated with a user's age and gender category to create a growing and increasingly accurate view of changes to senses by category.

What's more, results may be associated, anonymously, with users' genomics to see if there are correlations between sensory scores and specific genes. Where there are genetic predispositions to low auditory sense, for example, that information could help give medical personnel an early warning of impending hearing loss and an opportunity for intervention to correct it before it affects mental health and learning.

The embodiment shown and described is exemplary. External test kits may be modified and improved. Internal test displays and sounds for visual and auditory testing may also be modified to reduce differences and improve consistency.

Other programs may be added to the remote server that further analyze, compare, and provide additional sensory data scoring and categorization.

What is claimed is:

1. An application-guided human sense testing system comprising:
a remote sensor subsystem;
a mobile device subsystem;
a photoreception testing subsystem;
an audition testing subsystem;
an olfaction testing subsystem;
a gustation testing subsystem; and
a tactile reception subsystem.

2. A system as in claim 1 wherein:
the remote sensor subsystem comprises:
a central-processing subsystem;
a program memory subsystem;
a data memory subsystem;
at least one program;
a machine-learning module; and
a network interface subsystem.

3. A system as in claim 1 wherein:
the mobile device subsystem comprises:
a touch-screen subsystem;
a processing subsystem;
a program memory subsystem;
a data memory subsystem;
a visual display subsystem;
an auditory reproduction subsystem;
at least one program; and
a network interface subsystem.

4. A system as in claim 1 wherein:
the photoreception testing subsystem is stored and executed by the mobile device subsystem; and
photoreception testing results are stored by the mobile device subsystem and conveyed to the remote server subsystem via network.

5. A system as in claim 1 wherein:
the audition testing subsystem is stored and executed by the mobile device subsystem; and
audition testing results are stored by the mobile device subsystem and convened to the remote server subsystem via network.

6. A system as in claim 1 wherein:
the olfaction testing subsystem is external to the mobile device subsystem;
olfaction testing steps are guided by the mobile device subsystem; and
the olfaction testing step results are stored by the mobile device subsystem and conveyed to the remote server subsystem via network.

7. A system as in claim 1 wherein;
the gustation testing subsystem is external to the mobile device subsystem;
gustation testing steps are guided by the mobile device subsystem; and
the gustation testing step results are stored by the mobile device subsystem and conveyed to the remote server subsystem via network.

8. A system as in claim 1 wherein:
the tactile reception testing subsystem is external to the mobile device subsystem;
tactile reception testing steps are guided by the mobile device subsystem; and
the tactile reception testing step results are stored by the mobile device subsystem and conveyed to the remote server subsystem via network.

9. A system as in claim 1 wherein:
testing step results for photoreception, audition, olfaction, gustation and tactile reception are each compared to same-sense results, by the remote server, for all previously conveyed and stored results;
the remote server, using the at least one program and the machine-learning module, compares the testing step results, where those results are identified as to age and gender, with a subset of all the previously conveyed and stored results that match in age and gender; and
the remote server produces a dynamic set of same-age/same-gender results rankings wherein comparative rankings will change as more such results are received and stored.

10. A system as in claim 9 wherein;
the comparatively ranked testing step results, and same-age, same-gender ranks results determined by the remote server are conveyed by the remote server to the mobile device via network.
